(19) United States
(12) Patent Application Publication
(10) Pub. No.: US 2022/0230741 A1
(43) Pub. Date: Jul. 21, 2022

(54) Method for Remote Diagnostics and Vital Sign Monitoring

(71) Applicants: Michael Wynnik, Pelzer, SC (US); Darren Wynnik, Imperial, PA (US); George Richard Zimmerman, II, McMurray, PA (US)

(72) Inventors: Michael Wynnik, Pelzer, SC (US); Darren Wynnik, Imperial, PA (US); George Richard Zimmerman, II, McMurray, PA (US)

(21) Appl. No.: 17/405,102

(22) Filed: Aug. 18, 2021

Related U.S. Application Data
(60) Provisional application No. 63/139,100, filed on Jan. 19, 2021.

(51) Int. Cl. G16H 40/67; G16H 10/60; H04W 12/50

(52) U.S. Cl. CPC G16H 40/67; H04W 12/50; G16H 10/60

(57) ABSTRACT

The present invention relates to a system and method for receiving diagnostic and vital signs information remotely from a patient and providing treatment information to the patient. The system includes a plurality of wireless diagnostic tools to measure one or more vital signs of a patient, a computer implemented software platform configured to receive vital sign information from the one or more wireless diagnostic tools wirelessly or through manual input from a user. A healthcare provider system receives vital sign information of the patient from the computer implemented software through the internet. A doctor reviews the vital signs information of the patient and provides necessary advice to the patient. The system and method eliminate the need to physically visit a doctor's office for a simple physical or checkup and enables a healthcare provider to remotely obtain patient vital signs information.

### CROSS-REFERENCE TO RELATED APPLICATION

The present application claims priority to, and the benefit of, U.S. Provisional Application No. 63/139,100, which was filed on Jan. 19, 2021 and is incorporated herein by reference in its entirety.

### FIELD OF THE INVENTION

The present invention relates generally to the field of medical treatment systems. More specifically, the present invention relates to a telehealth interface system featuring several diagnostic tools to transmit vital sign information to a doctor over a software platform and a handheld touchscreen or remote device. The telehealth interface system features several diagnostic tools such as a stethoscope, heartbeat monitor, thermometer and other tools which are used to take vital sign readings for the doctor and are uploaded on the touchscreen or remote device. The telehealth interface system includes a software application which receives vital sign reading information through wireless connection or manual input from each of the diagnostic tools and allows the transmission of remote diagnostic and vital sign information from a patient to a healthcare provider in real time. The invention eliminates the need to physically visit a doctor's office for a simple physical checkup. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, people visit healthcare centers for routine physical checkups to get an overview of their overall health. At the healthcare centers, the doctors physically monitor the patient, and can use various diagnostic tools to analyze the patient's health and advise the patient accordingly. Regular health check-ups help the users to detect potentially life-threatening health issues early, before they cause a problem, and enables the user to get early treatment of their health conditions. Additionally, regular or routine health check-ups provide new ways to live a healthy, longer life, thereby improving the health of the individuals.

Some people may seek health advice and desire to visit healthcare centers even when they are suffering from mild symptoms of a disease, in order for the disease to be diagnosed timely and for proper medication to be administered promptly, thereby preventing them from suffering any life-threatening consequences of the disease. However, in times of a pandemic such as COVID-19, which is an epidemic of an infectious disease spreading worldwide and affecting millions of people, going to the doctor's office can be an anxiety-filled experience for some people. People may be worried about being infected by the virus of the pandemic or other infectious diseases while visiting healthcare centers. Harmful airborne pathogens capable of causing serious illness during a pandemic may cause people to be wary of visiting the doctor for a standard check-up. In such scenarios, if people do not get their health check-up in a timely manner, they may have to face more serious consequences later.

Individuals may seek solutions such as telehealth systems, which involve video conferencing for patient consultation, and allows patients to consult doctors for specific issues or symptoms. However, such telehealth systems are limited in diagnostics due to the lack of equipment on the patient's end. Due to such limited diagnostics, doctors may not be able to help patients in their full capacity. Additionally, the disease may not be properly diagnosed without proper testing and monitoring equipment. The currently available telehealth solutions fail to provide an efficient means for health check-ups and require the physical presence of patients in healthcare centers.

Therefore, there exists a long felt need in the art for a telehealth system that allows users to get their health check-ups done remotely, and that eliminates the need to physically visit healthcare centers for regular or routine health check-ups. Additionally, there is a long felt need in the art for a telehealth system that does not lack the appropriate diagnostic tools, allows monitoring of vital health parameters of users, and shares the monitored parameters with doctors for proper advice or diagnosis. Moreover, there is a long felt need in the art for a telehealth system which prevents individuals from visiting healthcare centers in the times of pandemics, and therefore protects them from any transmission of harmful pathogens causing infectious diseases. Further, there is a long felt need in the art for a telehealth system which provides a sense of relief to the users, as they do not have to visit healthcare centers during pandemics for regular check-ups. Finally, there is a long felt need in the art for an efficient telehealth system, with proper diagnostic tools, which enables a healthcare provider to remotely obtain patient vital sign information without their physical presence required.

The subject matter disclosed and claimed herein, in one embodiment thereof, comprises a method of remotely monitoring vital signs of a patient and providing treatment. The method comprising the steps of: monitoring one or more vital signs using one or more wireless diagnostic tools; the diagnostic tools include one or more of a wireless thermometer, a wireless stethoscope, a wireless pulse oximeter, a wireless heart monitor, a wireless blood pressure monitor and a wireless ear canal camera; each wireless diagnostic tool includes a wireless transmitter to wirelessly transmit the measured vital signs to a companion software application, wherein the software application is installed in an electronic device; displaying the received vital sign information by the software application; uploading the vital sign information of the patient along with the medical history to a healthcare provider server and to a monitoring system; inspecting the vital sign information of the patient along with a doctor or healthcare provider; and, contacting the patient by the doctor to provide advice and medication. The software application also compares the vital sign information with threshold values, and notifies the hospital system and emergency services in case any of the values are more than the threshold.

The subject matter disclosed and claimed herein, in another embodiment thereof, comprises a method of remotely monitoring vital signs of a patient and providing treatment. The method comprising the steps of: monitoring one or more vital signs using one or more electronically connectable diagnostic tools; the diagnostic tools include one or more of a thermometer, a stethoscope, a pulse oximeter, a heart monitor, a blood pressure monitor and an ear canal camera; each diagnostic tool includes an electronically connectable device to receive and transmit the measured vital signs to a companion software application, wherein the software application is installed in an electronic device; displaying the received vital sign information by the software application; uploading the vital sign information of the patient along with the medical history to a healthcare provider server and to a monitoring system; inspecting the vital sign information of the patient along with a doctor or healthcare provider; and, contacting the patient by the doctor to provide advice and medication. The software application also compares the vital sign information with threshold values and notifies the hospital system and emergency services in case any of the values are more than the threshold. The electronic connections can comprise a dongle to connect the diagnostic tools to a computer and the software application. It is to be appreciated that the dongle(s) connects to a port on the diagnostic tool to enable pass-through of information to the computer and subject software application. Alternatively, the electronic connections can comprise a USB, USB flash drive, or other cable to connect the diagnostic tools to the computer and the software application.

In this manner, the novel telehealth system of the present invention accomplishes all of the forgoing objectives, and provides a relatively safe, easy, convenient and efficient solution for allowing healthcare providers to remotely obtain patient's vital sign information without requiring the physical presence of the patients. The telehealth system of the present invention is also user friendly, as it eliminates the need to visit healthcare centers for routine/regular check-ups, and protects them from transmission of harmful pathogens and infectious diseases while visiting the doctors or healthcare centers.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a method of remotely monitoring the vital signs of a patient and subsequently providing treatment. The method comprises the steps of: monitoring one or more vital signs using one or more wireless diagnostic tools; the diagnostic tools include one or more of a wireless thermometer, a wireless stethoscope, a wireless pulse oximeter, a wireless heart monitor, a wireless blood pressure monitor and a wireless ear canal camera; each wireless diagnostic tool includes a wireless transmitter to wirelessly transmit the measured vital signs to a companion software application wherein the software application is installed in an electronic device; displaying the received vital sign information by the software application; uploading the vital sign information of the patient along with the medical history to a healthcare provider server and to a monitoring system, inspecting the vital sign information of the patient by a doctor of the healthcare provider; and, contacting the patient by the doctor to provide advice and medication. The software application also compares the vital sign information with threshold values, and notifies the hospital system and emergency services in case any of the values are more than the threshold.

The subject matter disclosed and claimed herein, in another embodiment thereof, comprises a method of remotely monitoring vital signs of a patient and providing treatment. The method comprising the steps of: monitoring one or more vital signs using one or more electronically connectable diagnostic tools; the diagnostic tools include one or more of a thermometer, a stethoscope, a pulse oximeter, a heart monitor, a blood pressure monitor and an ear canal camera; each diagnostic tool includes an electronically connectable device to receive and transmit the measured vital signs to a companion software application, wherein the software application is installed in an electronic device; displaying the received vital sign information by the software application; uploading the vital sign information of the patient along with the medical history to a healthcare provider server and to a monitoring system; inspecting the vital sign information of the patient along with a doctor or healthcare provider; and, contacting the patient by the doctor to provide advice and medication. The software application also compares the vital sign information with threshold values and notifies the hospital system and emergency services in case any of the values are more than the threshold. The electronic connections can comprise a dongle to connect the diagnostic tools to a computer and the software application. It is to be appreciated that the dongle(s) connects to a port on the diagnostic tool to enable pass-through of information to the computer and subject software application. Alternatively, the electronic connections can comprise a USB, USB flash drive, or other cable to connect the diagnostic tools to the computer and the software application.

In a further embodiment of the present invention, a remote diagnostic and vital sign information system is disclosed and comprises: a plurality of wireless diagnostic tools to measure the vital signs of a patient; the diagnostic tools include one or more of a wireless thermometer, a wireless stethoscope, a wireless pulse oximeter, a wireless heart monitor, a wireless blood pressure monitor and a wireless ear canal camera; a computer-implemented software installed in an electronic device which causes the electronic device to receive the vital signs from one or more diagnostic tools, and create graphs to represent the received vital signs; and upload the vital signs to a healthcare service provider system. One or more of body temperature, heart rate, SpO2, blood pressure, ear canal information or pulse rate is measured by the diagnostic tools, and is transmitted to the computer-implemented software. The healthcare system and an emergency service can receive a notification if the vital signs received by the computer-implemented software are above or below the threshold values for each vital sign.

In yet a further embodiment of the present invention, a remote vital sign monitoring system is disclosed and comprises: a plurality of wireless diagnostic tools to measure the vital signs of a patient, wherein each wireless diagnostic tool wirelessly transmits the measured vital sign; a computer-implemented software installed in an electronic device, which causes a process of the electronic device to receive the vital signs from one or more diagnostic tools, and create graphs to represent the received vital signs; and, a healthcare service provider system and database to receive vital signs from the computer-implemented software.

In yet still a further embodiment of the present invention, a method of remotely monitoring and communicating vital signs from an individual is disclosed. The method comprising: pairing user devices associated with a first user, a second user, and at least a third user with a remote healthcare monitoring app; monitoring a plurality of vital signs of the first user using at least one diagnostic tool; electronically transmitting the plurality of vital signs to the first user of the healthcare monitoring app; displaying the plurality of vital signs on the first user app; uploading the plurality of vital signs to the second user of the healthcare monitoring app, wherein the second user is a caregiver; uploading the plurality of vital signs to the third user of the healthcare monitoring app, wherein the third user is a designated family member of the first user; inspecting the plurality of vital signs by the caregiver; contacting the first user by the caregiver; and advising the first user by the caregiver.

In another embodiment of the present invention, a method of remotely monitoring and communicating vital signs from an individual is disclosed. The method comprising: pairing user devices associated with a first user, a second user and at least a third user with a remote healthcare monitoring app; monitoring a plurality of vital signs of the first user using at least one diagnostic tool; electronically transmitting the plurality of vital signs to the first user of the healthcare monitoring app; displaying the plurality of vital signs on the first user app; uploading the plurality of vital signs to the second user of the healthcare monitoring app, wherein the second user is a caregiver; inspecting the plurality of vital signs by the caregiver; contacting the first user by the caregiver; advising the first user by the caregiver; wherein the monitoring of the plurality of vital signs includes at least one electronically connected diagnostic tool selected from a group consisting of a thermometer, a stethoscope, a pulse oximeter, a heart monitor, a blood pressure monitor and an ear canal camera; comparing the plurality of vital signs of the first user with respective normal vital sign ranges for the plurality of vital signs; notifying the second user and the third user when one of the plurality of vital signs is out of range of the normal vital sign ranges for the plurality of vital signs; and uploading the plurality of vital signs to the third user of the healthcare monitoring app, wherein the third user is a designated family member of the first user.

The advantage of the system of the present invention is that it provides users with a telehealth interface system featuring several diagnostic tools to transmit vital sign information to a doctor over a software platform and a handheld touchscreen device. The system enables a healthcare provider or doctor to remotely obtain patient vital sign information without being physically present near the patient. The system eliminates the need for a patient to physically visit a doctor's office for a simple physical or check-up. Patients can receive remote health consultations and information from their doctor without having to physically visit the office.

Additionally, a high or low value (i.e. thresholds) for vital signs of a patient can alert a remote healthcare provider as to who can receive alerts over the Internet or urgent notifications over the phone. Reports of health/activity indicators and the overall well-being of the patient can be compiled in a medical history profile for viewing by a remote healthcare provider. Feedback reports can be sent to the patients, their designated informal caregiver and their remote healthcare provider. The content of the report can be tailored to the target recipient's needs, and can present the information in a format understandable by an elderly person unfamiliar with computers, via an appealing patient interface. The remote healthcare provider will have access to the health and well-being status of their patients without being intrusive, having to call, or personally visit to obtain an individual's medical history profile.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of one potential embodiment of the remote diagnostic and vital sign information system of the present invention in accordance with the disclosed architecture;

FIG. 2 illustrates a perspective view showing various components available to a patient for monitoring vital sign information using one potential embodiment of the remote diagnostic and vital signs information system of the present invention in accordance with the disclosed architecture;

FIG. 3 illustrates a flow diagram showing the steps of measuring vital sign information of a patient and uploading the vital sign information to a healthcare provider system using one potential embodiment of the remote diagnostic and vital sign information system of the present invention in accordance with the disclosed architecture;

FIG. 4 illustrates a flow diagram showing the steps of transmitting vital sign information and receiving medication and other treatment remotely, using one potential embodiment of the remote diagnostic and vital sign information system of the present invention in accordance with the disclosed architecture;

FIG. 5 illustrates a block diagram showing modules of the smartphone application or a software module used in conjunction with one potential embodiment of the remote diagnostic and vital sign information system of the present invention in accordance with the disclosed architecture;

FIG. 6 illustrates a block diagram showing modules of an exemplary diagnostic tool and of an electronic device in which the software application is installed;

FIG. 7 illustrates a user using diagnostic tools available in the remote diagnostic and vital sign information system to measure the vital sign information in accordance with the disclosed architecture;

FIG. 8 illustrates vital sign information displayed on the software application or module installed in a smartphone in accordance with the disclosed architecture; and

FIG. 9 illustrates vital sign information of a patient viewed by a doctor on a display device in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It can be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention, and do not limit the scope of the invention. Additionally, an illustrated embodiment need not have all the aspects or advantages shown. Thus, in other embodiments, any of the features described herein from different embodiments can be combined.

As noted above, there exists a long felt need in the art for a telehealth system which allows users to get their health check-ups done remotely. There is also a long felt need in the art for a telehealth system which eliminates the need to physically visit the healthcare centers for regular or routine health check-ups. Additionally, there is a long felt need in the art for telehealth systems that conveniently provide diagnostic tools, and allow the monitoring of vital health parameters of the users, and sharing of the monitored parameters with doctors for proper advice or suggestions. Moreover, there is a long felt need in the art for telehealth systems which prevent individuals from visiting healthcare centers in the times of pandemics, and therefore protects them from any transmission of harmful pathogens causing infectious diseases. Further, there is a long felt need in the art for telehealth systems which provide a sense of relief to the users as they do not have to visit healthcare centers during pandemics for regular check-ups. Finally, there is a long felt need in the art for an efficient telehealth system with proper diagnostic tools, which enables a healthcare provider to remotely obtain patient vital sign information without a physical presence required.

The present invention, in one exemplary embodiment, is a novel method of remotely monitoring the vital signs of a patient and subsequently providing treatment. The method comprising: monitoring one or more vital signs using one or more wireless diagnostic tools; the diagnostic tools include one or more of a wireless thermometer, a wireless stethoscope, a wireless pulse oximeter, a wireless heart monitor, a wireless blood pressure monitor and a wireless ear canal camera; each wireless diagnostic tool includes a wireless transmitter to wirelessly transmit the measured vital signs to a companion software application, wherein the software application is installed in an electronic device; displaying the received vital signs information by the software application; uploading the vital sign information of the patient along with the medical history to a healthcare provider server and to a monitoring system; inspecting the vital sign information of the patient by a doctor of the healthcare provider; and, contacting the patient by the doctor to provide advice and medication. The software application also compares the vital sign information with threshold values or ranges, and notifies hospital system and emergency services in case any of the values are more or less than the threshold ranges.

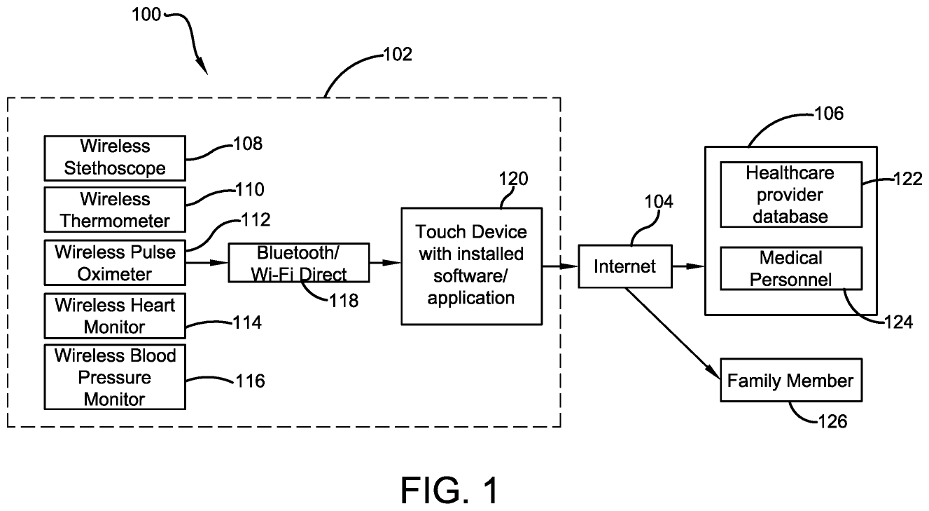

Referring initially to the drawings, FIG. 1 illustrates a schematic view of a remote diagnostic and vital sign information system of the present invention as per the disclosed architecture. The remote diagnostic and vital sign information system 100 of the present invention transmits the vital sign information from a patient at home to a health care provider in real time (i.e. simultaneously with the monitoring) and remotely. Additionally, the system 100 can also transmit the information to a family member or caregiver. The system 100 includes several integrated diagnostic tools, with a wireless transmitter for connection to an electronic device, such as a touch device. A computer-implemented application is installed in the electronic device to receive information from the diagnostic tools and upload the information to a healthcare provider over the internet.

As shown, a plurality of diagnostic tools are present and available to a patient 102. The plurality of diagnostic tools are configured to measure the vital signs of a user, each comprising a wireless transmitter, dongle, USB, USB flash drive, or other electronic connection. The diagnostic tools can be included in a kit available commercially to a user or a patient, and can be available as a part of subscription services offered by a healthcare provider. The diagnostic tools can include a wireless stethoscope 108, a wireless thermometer 110, a wireless pulse oximeter 112, a wireless heart monitor patch 114 and a wireless blood pressure monitor 116. The diagnostic tools can include any additional and alternative wireless vital sign detectors. Based on the subscription services adopted by the patient, the diagnostic tools can include or exclude any vital sign detecting device.

It should be noted that in one potential embodiment of the present invention, all the diagnostic tools have wireless transmitters to wirelessly transmit the detected vital signs to the electronic device 120 having the installed application. Alternatively, the diagnostic tools may not be equipped with a wireless transmitter, and in such cases, a user can manually input the recorded vital sign information into the software application, using the input device such as a touchpad of the electronic device 120. In another embodiment of the present invention, all the diagnostic tools can include a dongle, USB, USB flash drive, or other electronic connection to pass-through the respective data (i.e. vital signs) from the diagnostic tool to the electronic device 120 having the installed application.

Once the vital sign information of a user is detected using one or more diagnostic tools, the vital signs can be transmitted wirelessly in real time to the paired smartphone application or software module installed in the electronic device 120. A wireless channel 118 such as a Bluetooth channel or a Wi-Fi Direct or Wi-Fi channel is established with the electronic device 120 and the diagnostic tools. The received vital sign information is displayed on the display device of the electronic device 120 for actions and uploaded to a healthcare provider.

When the diagnostic tools are turned on, a wireless connection 118 is automatically set up between the diagnostic tool and the electronic device 120. A wireless transmitter of each of the diagnostic tools establishes the wireless connection 118 with the wireless transmitter/receiver of the electronic device.

The electronic device 120, using the wireless transmitter, transmits the received vital sign information to a healthcare provider 106 network. The information is transmitted using a network connection, such as an internet 104 connection, to a healthcare provider database 122 and medical personnel 124. The patient can be given real-time expert advice and treatment without requiring the patient to go to a doctor's office. Based on the vital sign information, an appropriate medical expert can contact the patient. Additionally, the vital sign information can also be transmitted to a family member 120, or to any caretaker.

The remote diagnostic and vital sign information system 100 of the present invention provides users with a telehealth interface system featuring several diagnostic tools to transmit vital sign information to a doctor over a software platform, and a handheld touchscreen device 120. The system includes a software application capable of receiving information through wireless connections from each of the diagnostic tools or through a manual input. The diagnostic tools are easy to use, and can be used by a person at home. The system features several diagnostic tools such as a stethoscope, heartbeat monitor, thermometer and other tools used to take vital readings for sharing with the doctor.

The network 104 is an electronic communication network that facilitates communication between the electronic device 120, and the healthcare provider system 106. The network 104 can include routers, switches, mobile access points, bridges, hubs, intrusion detection devices, storage devices, standalone server devices, desktop computers, firewall devices, laptop computers, handheld computers and other types of computing devices. The network 104 can be implemented as one or more local area networks (LANs), metropolitan area networks, subnets, wide area networks (such as the Internet), or can be implemented at another scale.

Wireless diagnostic devices are configured to attach to, be positioned in contact with or otherwise be directly connected to a patient for monitoring vital signs. The diagnostic tools acquire one or more vital pieces of information. The vital data of a patient, such as heart rate, temperature, SpO2 level etc. is acquired and transmitted to healthcare providers.

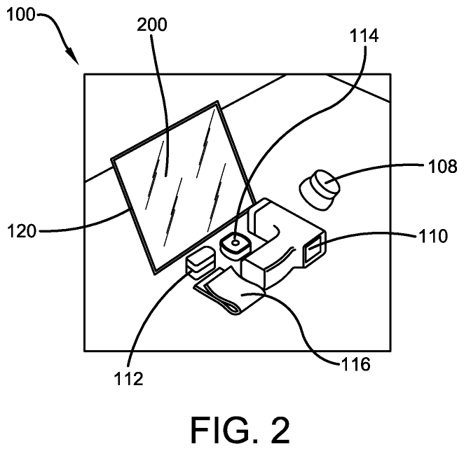

FIG. 2 illustrates a perspective view showing components available to a patient for monitoring vital sign information using the remote diagnostic and vital sign information system of the present invention. The diagnostic tools provided to a patient include a wireless stethoscope 108, a wireless thermometer 110, a wireless pulse oximeter 112, a wireless heart monitor 114 and a wireless blood pressure monitor 116. It is to be appreciated that wireless comprises dongles, USB flash drives, and other electronic data communication devices.

The wireless stethoscope 108 can be an electronic stethoscope which is able to transmit auscultation to a companion software application 200 installed in an electronic device 120. The stethoscope 108 can connect wirelessly to a smart device to transmit auscultations. The wireless thermometer 110 measures the body temperature of a patient using infrared technology, and transmits the recorded temperature value to the companion software application 200 installed in an electronic device 120. The wireless thermometer 110 can also have a display to show the recorded values.

The wireless pulse oximeter 112 records SpO2 and pulse rate of a patient. The oximeter 112 can also have a display to show the recorded values. The oximeter 112 connects wirelessly to the electronic device 120 to transmit the recorded values. The oximeter 112 touches the skin of a fingertip of the patient. The wireless heart monitor 114 is applied to the chest area of a user and is a next-generation cardiac monitoring device. The heart monitor 114 uses electrodes and a recording device to track the heart's rhythm and transmit wirelessly to the electronic device 120.

The wireless blood pressure monitor 116 provides high accuracy blood pressure monitoring and detailed results, and displays recommended values (i.e. ranges) in the application 200 installed in the electronic device 120. The blood pressure monitor 116 is placed on the arm. The application can store all the readings as part of a patient's medical history profile.

The touch device 120 can be provided by a healthcare provider, or alternatively the smartphone application 200, can be installed in a conventional smartphone or any other electronic device. The smartphone application 200 can be downloaded from a mobile application store. The touch device 120, if provided by the healthcare provider or acquired in a subscription plan, can be available as an application (i.e. app) 200 and pre-installed on the touch device 120.

A profile of a patient including personal details and medical history can be created and communicated with the healthcare provider. The vital sign information received by the smartphone application 200 can be uploaded to the healthcare provider network using internet or any other communication network and linked to the patient profile for a doctor to correlate the information with the medical history of the patient in order to provide comprehensive medical advice. It is to be appreciated that the medical history profile of a user can include recording multiple historical monitors of each of the plurality of vital signs of the user. The vital sign information can be inputted into the smartphone application 200 by: wireless transmission of each of the individual diagnostic tools; transfer through a dongle or flash drive; or, manual entry by a patient in case the diagnostic tools do not have wireless transmitters.

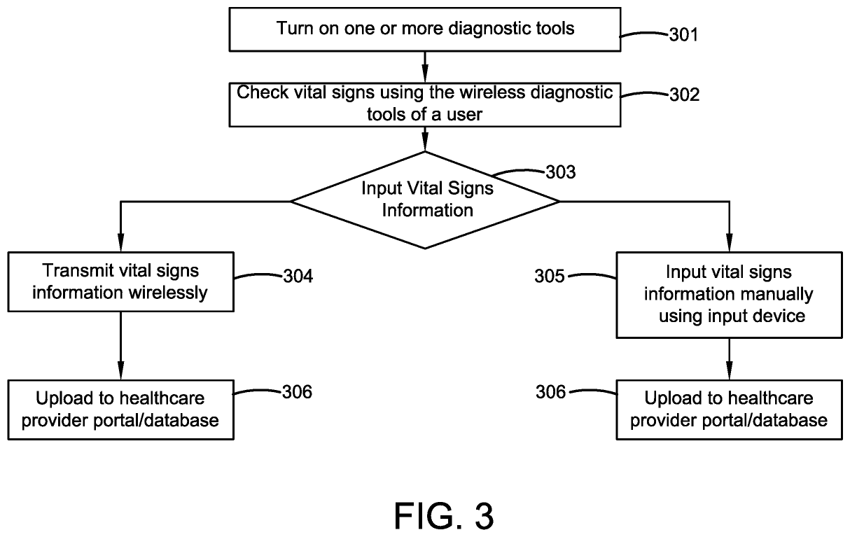

FIG. 3 illustrates a flow diagram showing steps of measuring vital sign information of a patient, and uploading the vital sign information to a healthcare provider system. Initially, one or more diagnostic tools are turned on for detection of the vital signs of a patient (Block 301). Then, one or more vital signs of the patient are measured using the diagnostic tools (Block 302). Alternative ways to input the vital sign information to a companion smartphone application or software module are determined by the patient (Block 303). If the diagnostic tools have wireless transmitters, then the vital signs are transmitted to the companion smartphone application or software module wirelessly and in real time (Block 304). If the diagnostic tools do not have wireless transmitters, then the vital signs are inputted in the companion smartphone application or software module manually by a user (Block 305). In both the cases, using an “Upload header”, the vital signs are uploaded to the healthcare provider system (Block 306).

A patient's information in the healthcare system is updated with the most recent vital sign information of the patient. The diagnostic tools and the smartphone application/software module eliminates the need for a patient to physically visit a doctor's office for a simple physical or check-up. The healthcare provider can remotely obtain patient vital sign information without a physical check-up.

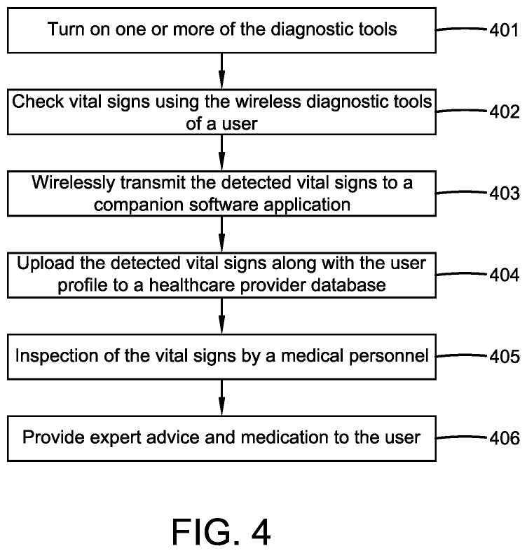

FIG. 4 illustrates a flow diagram showing the steps of transmitting vital sign information and receiving medication and other treatment remotely, using the remote diagnostic and vital sign information system of the present invention. As stated earlier, the patient can share the vital sign information and other health-related data with a doctor remotely and need not visit the doctor physically. This is helpful in the times of a pandemic, and also for elderly people who find it difficult to physically visit a doctor.

In the present embodiment, a patient uses wireless diagnostic tools to measure vital sign information and wirelessly transmit the vital sign information. Initially, one or more wireless diagnostic tools are turned on by a patient (Block 401). By direct or indirect physical contact or connection, the vital signs of a patient are measured (Block 402). Vital signs such as body temperature, heart rate, SpO2, etc. can be measured using the diagnostic tools. Then, the detected vital signs of the patient are wirelessly transmitted to a companion smartphone application or software module (Block 403). Using the companion smartphone application or software module, the detected vital signs, along with the user profile or medical history profile, are uploaded to a healthcare provider database (Block 404). Once uploaded, the vital signs are reviewed by a doctor (Block 405). The doctor can be a personal doctor of the patient, or can be assigned based on the medical profile of the patient. The doctor then provides expert advice, medication, and/or prescription recommendations to the patient (Block 406). The patient can also chat with the doctor on a phone call, or on a chat portal provided on the application.

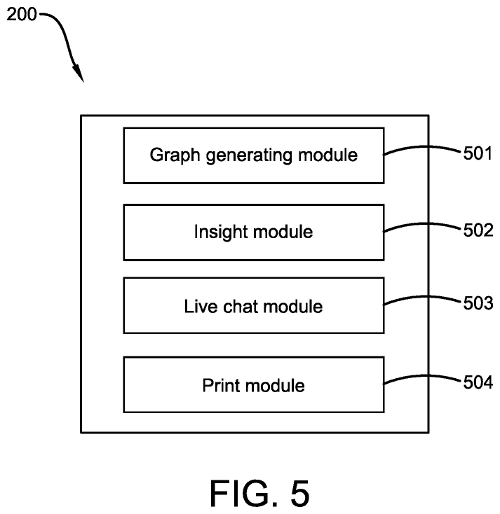

FIG. 5 illustrates a block diagram showing modules of the smartphone application or a software module used in the remote diagnostic and vital sign information system of the present invention. The software application 200 can be pre-installed in a touch device, or can be installed from an application (i.e. app) store. The software application 200 displays the vital sign information in digital form. A graph-generating module 501 creates graphs of various types to show the real-time vital sign information along with the historical (i.e. medical history profile) vital values of the patient. Comparison with the baseline and threshold values or ranges can also be displayed.

An insight module 502 displays insights based on the recorded values and the threshold values. Necessary first aid can be administered and direct contact to an emergency service or a caretaker of the patient can be accomplished. A live chat module 503 allows the patient to contact an available doctor to consult and discuss on a video call service offered by the smartphone application or a software module 200. The graphs and digital values displayed on the screen of the electronic device or a touch device can be printed or downloaded using a print module 504. It should be noted that the software application 200 can access different components such as a camera, input device, wireless transmitter and receiver of the electronic device 120, in which the software application 200 is installed.

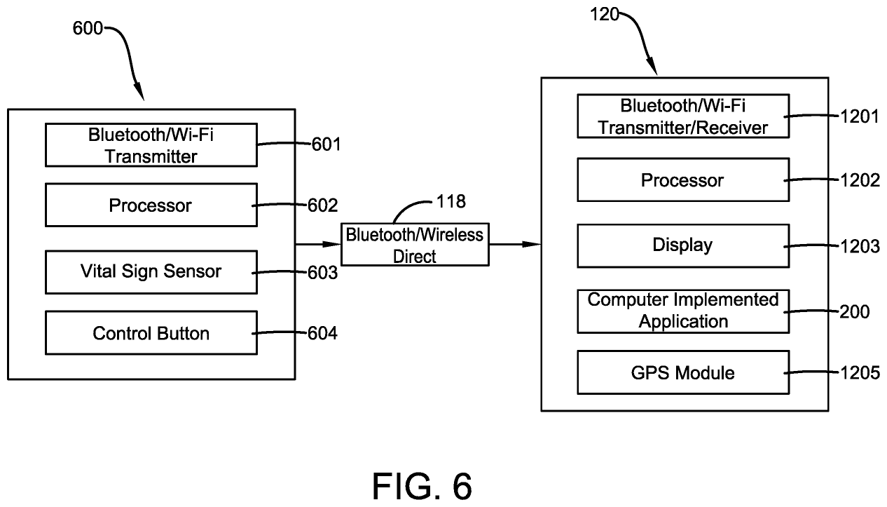

FIG. 6 illustrates a block diagram showing modules of an exemplary diagnostic tool and of an electronic device in which the software application is installed. The exemplary diagnostic tool 600 represents any of the diagnostic tools described above, or any other diagnostic tool that can be used within the system of the present invention. The diagnostic tool 600 includes a wireless transmitter 601, which can be used for transmitting the recorded vital values. A processor 602 for controlling the operations of the components, and also to process the recorded values is also present. A vital sign sensor 603 is present to record and detect specific vital sign information of a patient. In each diagnostic tool, a different vital sign sensor is present to record the respective vital sign information.

The electronic or touch device 120 includes at least a Bluetooth/Wi-Fi Transmitter/Receiver 1201, a processor 1202, a display device 1203 such as a an LED or LCD touch screen and a GPS module 1205. The software module 200 installed in the electronic or touch device 120 uses the Bluetooth/Wi-Fi Transmitter/Receiver 1201 to receive vital sign information from the sensor devices 600 via wireless connection 118, and uploads the same to the healthcare provider system using a communication network, such as internet.

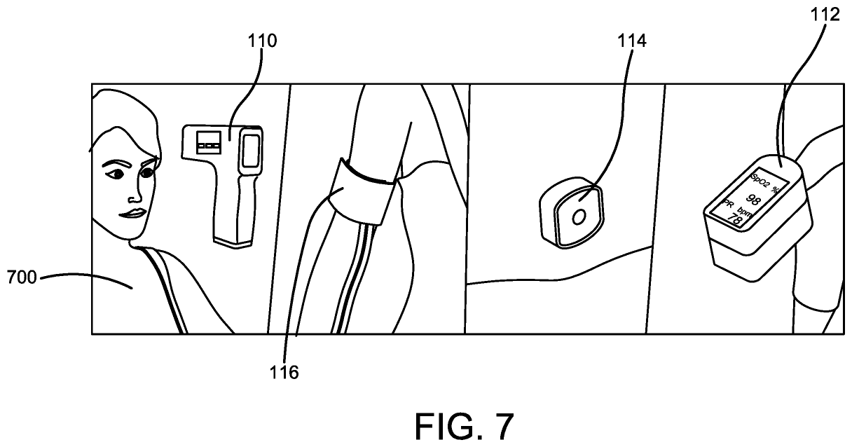

FIG. 7 illustrates a patient using diagnostic tools available in the remote diagnostic and vital sign information system to measure vital sign information in accordance with the disclosed architecture. As shown, a user 700 uses a wireless thermometer 110 to measure body temperature using infrared technology. The blood pressure monitor 116 is placed on the arm and provides highly accurate blood pressure values. The wireless heart monitor 114 is a next-generation cardiac monitoring device, which is applied to the chest of the user. The heart monitor 114 uses electrodes and a recording device to track the heart's rhythm. The wireless pulse oximeter 112 records SpO2 and the pulse rate of a patient. The oximeter 112 can also have a display to show the recorded values. All the diagnostic tools can have wireless transmitters, and can wirelessly transmit the measured vital signs information of the user 700 to the software application installed in an electronic device.

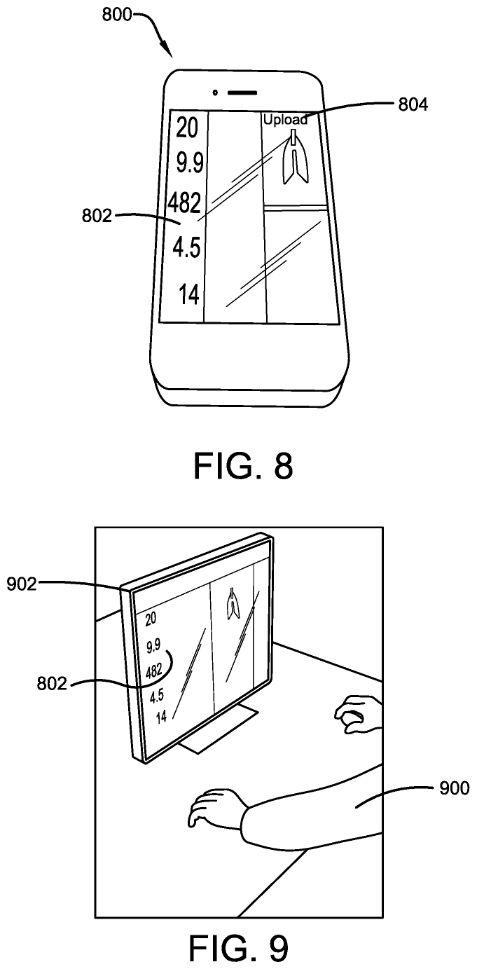

FIG. 8 illustrates vital sign information displayed on the software application or module installed in a smartphone in accordance with the disclosed architecture. In the present embodiment, a smartphone 800 displays the graphs and digital values 802 to indicate the vital sign information and using the “Upload header” 804, the information can be uploaded to a healthcare provider.

FIG. 9 illustrates vital sign information of a patient viewed by a doctor on a display device in accordance with the disclosed architecture. The vital sign information uploaded by a patient is viewed and evaluated by a doctor 900, who is remotely situated relative to the patient. The doctor 900 can view and evaluate the vital sign information 802, along with the graphs and medical history profile of the patient on a display device 902. The doctor can contact the patient, and upload the necessary medication, prescription, and medical advice which can then be received on the software application by the patient.

All the vital sign information of the patient is recorded in one or more healthcare provider databases. The vital sign information can be supplemented with reports from CT scans, X-rays, ultrasound scans and other similar medical tests and procedures. In one embodiment, a patient can book an appointment for a medical test using the software application. The software application can also contact emergency services and family members of the patient in case the vital sign data reveals an emergency situation. The healthcare system can also receive a notification in such cases, for example, to cause a nurse or doctor responsible for the patient to either monitor the progress of the condition or to take the appropriate medical actions.

One skilled in the art would understand in view of the present disclosure, that various systems and methods described above provide a number of important benefits to the remote vital-sign monitoring system of the present disclosure. The system of the present invention can exhibit significantly higher life-expectancy of patients. Whenever vital sign values exceed a threshold limit, the vital-sign values can be shown on a display terminal of the hospital system, along with the location and other profile details of the patient.

Certain terms are used throughout the following description and claims to refer to particular features or components. As one skilled in the art will appreciate, different persons can refer to the same feature or component by different names. This document does not intend to distinguish between components or features that differ in name but not structure or function. As used herein “remote diagnostic and vital signs information system”, “telehealth interface system” and “telehealth system” are interchangeable and refer to the remote diagnostic and vital sign information system 100 of the present invention.

Notwithstanding the forgoing, the remote diagnostic and vital sign information system 100 of the present invention can be of any suitable size and configuration as is known in the art, without affecting the overall concept of the invention, provided that it accomplishes the above-stated objectives. One of ordinary skill in the art will appreciate that the size, configuration and material of the remote diagnostic and vital sign information system 100 as shown in the FIGS. are for illustrative purposes only, and that many other sizes and shapes of the remote diagnostic and vital sign information system 100 are well within the scope of the present disclosure. Although the dimensions of the remote diagnostic and vital sign information system 100 are important design parameters for user convenience, the remote diagnostic and vital sign information system 100 can be of any size that ensures optimal performance during use, and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art can recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method of remotely monitoring and communicating vital signs from an individual, the method comprising the steps of:
pairing user devices associated with a first user, a second user, and at least a third user with a remote healthcare monitoring app;
monitoring a plurality of vital signs of said first user using at least one diagnostic tool;
electronically transmitting said plurality of vital signs to said first user of said healthcare monitoring app;
displaying said plurality of vital signs on said first user app;
uploading said plurality of vital signs to said second user of said healthcare monitoring app, wherein said second user is a caregiver;
uploading said plurality of vital signs to said third user of said healthcare monitoring app, wherein said third user is a designated family member of said first user;
inspecting said plurality of vital signs by said caregiver;
contacting said first user by said caregiver; and
advising said first user by said caregiver.

2. The method of claim 1, wherein said monitoring of said plurality of vital signs includes said at least one diagnostic tool selected from a group consisting of a thermometer, a stethoscope, a pulse oximeter, a heart monitor, a blood pressure monitor, and an ear canal camera.

3. The method of claim 2, further comprising simultaneously contacting said first user and said third user by said caregiver.

4. The method of claim 2, wherein said transmitting said plurality of vital signs is real time with said monitoring of said plurality of vital signs.

5. The method of claim 1, further comprising comparing said plurality of vital signs of said first user with respective normal vital sign ranges for said plurality of vital signs.

6. The method of claim 5, further comprising notifying said second user and said third user when one of said plurality of vital signs are out of range of said normal vital sign ranges for said plurality of vital signs.

7. The method of claim 5 further comprising building a medical history profile of said first user, wherein said medical history profile includes recording multiple historical monitors of each of said plurality of vital signs of said first user.

8. The method of claim 7 further comprising graphing said medical history profile and said plurality of vital signs of said first user.

9. The method of claim 8, further comprising comparing said medical history and said plurality of vital signs of said first user with said respective normal vital sign ranges for said plurality of vital signs.

10. The method of claim 9, further comprising uploading prescription information and medical advice by said caregiver and transmitting said prescription information and said medical advice to said first user.

11. A method of remotely monitoring and communicating vital signs from an individual, the method comprising:
pairing user devices associated with a first user, a second user, and at least a third user with a remote healthcare monitoring app;
monitoring a plurality of vital signs of said first user using at least one diagnostic tool;
transmitting wirelessly said plurality of vital signs to said first user of said healthcare monitoring app;
displaying said plurality of vital signs on said first user app;
uploading said plurality of vital signs to said second user of said healthcare monitoring app, wherein said second user is a caregiver;
inspecting said plurality of vital signs by said caregiver;
contacting said first user by said caregiver;
advising said first user by said caregiver;
wherein said monitoring of said plurality of vital signs includes at least one wireless diagnostic tool selected from a group consisting of a thermometer, a stethoscope, a pulse oximeter, a heart monitor, a blood pressure monitor, and an ear canal camera;
comparing said plurality of vital signs of said first user with respective normal vital sign ranges for said plurality of vital signs;
notifying said second user and said third user when one of said plurality of vital signs are out of range of said normal vital sign ranges for said plurality of vital signs; and
uploading said plurality of vital signs to said third user of said healthcare monitoring app, wherein said third user is a designated family member of said first user.

12. The method of claim 11, further comprising simultaneously contacting said first user and said third user by said caregiver.

13. The method of claim 11, wherein said transmitting said plurality of vital signs is real time with said monitoring of said plurality of vital signs.

14. The method of claim 11, further comprising building a medical history profile of said first user, wherein said medical history profile includes recording multiple historical monitors of each of said plurality of vital signs of said first user.

15. The method of claim 14, further comprising graphing said medical history profile and said plurality of vital signs of said first user.

16. The method of claim 15, further comprising comparing said medical history and said plurality of vital signs of said first user with said respective normal vital sign ranges for said plurality of vital signs.

17. The method of claim 16, further comprising uploading prescription information and medical advice by said caregiver and transmitting said prescription information and said medical advice to said first user.

18. The method of claim 17 further comprising a step of combining said plurality of vital signs with other medical test results in a first user healthcare database.

19. The method of claim 18, wherein said other medical test results are selected from the group consisting of CT scan, X-RAY scan, and ultrasound.

20. The method of claim 16 further comprising a step of simultaneously contacting said first user, said third user, and emergency services when said comparing said plurality of vital signs of said first user with said respective normal vital sign ranges for said plurality of vital signs reveals an emergency situation.
